(19) United States
(12) Patent Application Publication
(10) Pub. No.: US 2024/0079121 A1
(43) Pub. Date: Mar. 7, 2024

(54) WORKFLOW AUTOMATION EXECUTION METHOD APPLIED IN CLOUD-NATIVE BIOMEDICAL INFORMATICS PLATFORM AND SYSTEM THEREOF, AND COMPUTER READABLE MEDIUM

(71) Applicant: Atgenomix Incorporated, Taipei City (TW)

(72) Inventors: Ming-Tai CHANG, Taipei City (TW); Yun Lung LI, Taipei City (TW)

(21) Appl. No.: 18/457,334

(22) Filed: Aug. 28, 2023

Related U.S. Application Data
(60) Provisional application No. 63/373,858, filed on Aug. 29, 2022.

(51) Int. Cl. G16H 40/20; H04L 67/10

(52) U.S. Cl. CPC G16H 40/20; H04L 67/10

(57) ABSTRACT

A TRS workflow is registered in a cloud-native biomedical informatics platform, and the TRS workflow includes at least one TRS_FQN. A workflow automation execution method applied in the cloud-native biomedical informatics platform includes providing a run sheet used to configure a sequencer to generate a first sequencing data. The run sheet includes a sample-specific metadata associating the first sequencing data, at least one TRS_FQN and a run identifier. The method further includes importing a first DRS object indicating the first sequencing data in response to the run sheet. A first WES_FQN is generated based on the run identifier and the at least one TRS_FQN and attached to the first DRS object. The method further includes performing a WES run running the TRS workflow on the first DRS object in response to the run sheet. The first DRS object is resolved based on the first WES_FQN.

### RELATED APPLICATIONS

This application claims priority to U.S. Provisional Application Ser. No. 63/373,858, filed Aug. 29, 2022, which is herein incorporated by reference.

### BACKGROUND

### Technical Field

The present disclosure relates to a workflow automation execution method and a system thereof, and a computer readable medium. More particularly, the present disclosure relates to a workflow automation execution method applied in a cloud-native biomedical informatics platform and a system thereof, and a computer readable medium.

### Description of Related Art

Whole genome sequencing, such as Next-generation sequencing (NGS), is progressively more applied to biomedical research, clinical, and personalized medicine applications to identify disease-associated and/or drug-associated genetic variants to advance precision medicine. The impact of NGS technologies in revolutionizing the biological and clinical sciences has been unprecedented.

Post-sequencing DNA analysis typically includes read mapping, variant calling, and annotation. The analysis workflows as a whole is very time-consuming and computationally, especially for whole genome sequencing. With the ever increasing rate at which next-generation sequencing (NGS) data is generated, it is important to improve the data processing and analysis workflow.

As the complexity of an individual workflow increases to handle a variety of use cases, it becomes more challenging to optimally compute with it. For example, analyses may incorporate nested workflows, business logic, memoization, parallelization, the ability to restart failed workflows, or require parsing of metadata—all of which compound the challenges in optimizing workflow execution. Further, increases in complexity make it challenging to port computational workflows to different environments or systems. As a result of the increasing volume of biomedical data, analytical complexity, and the scale of collaborative initiatives focused on data analysis, reliable and reproducible analysis of biomedical data has become a significant concern. Accordingly, there is a need for improvements in computational workflow execution.

### SUMMARY

According to one aspect of the present disclosure, a tool register service (TRS) workflow is registered in a cloud-native biomedical informatics platform, and the TRS workflow includes at least one tool register service fully qualified name (TRS_FQN). A workflow automation execution method applied in the cloud-native biomedical informatics platform includes providing a run sheet used to configure a sequencer to generate a first sequencing data, wherein the run sheet includes a sample-specific metadata associating the first sequencing data, the at least one TRS_FQN and a run identifier; importing a first data repository service (DRS) object indicating the first sequencing data in response to the run sheet, wherein a first workflow execution service fully qualified name (WES_FQN) is generated based on the run identifier and the at least one TRS_FQN and attached to the first DRS object; and performing a workflow execution service (WES) run running the TRS workflow on the first DRS object in response to the run sheet, wherein the first DRS object is resolved based on the first WES_FQN.

According to another aspect of the present disclosure, a tool register service (TRS) workflow is registered in a cloud-native biomedical informatics platform, and the TRS workflow includes at least one tool register service fully qualified name (TRS_FQN). A workflow automation execution system includes a memory and a processor. The memory stores a run sheet and a first sequencing data. The processor is signally connected to the memory and configured to perform a workflow automation execution method applied in the cloud-native biomedical informatics platform. The workflow automation execution method applied in the cloud-native biomedical informatics platform includes providing the run sheet used to configure a sequencer to generate the first sequencing data, wherein the run sheet includes a sample-specific metadata associating the first sequencing data, the at least one TRS_FQN and a run identifier; importing a first data repository service (DRS) object indicating the first sequencing data in response to the run sheet, wherein a first workflow execution service fully qualified name (WES_FQN) is generated based on the run identifier and the at least one TRS_FQN and attached to the first DRS object; and performing a workflow execution service (WES) run running the TRS workflow on the first DRS object in response to the run sheet, wherein the first DRS object is resolved based on the first WES_FQN.

According to further another aspect of the present disclosure, a computer readable medium has instructions therein, when executed, causing a processor to perform a workflow automation execution method applied in a cloud-native biomedical informatics platform. A tool register service (TRS) workflow is registered in the cloud-native biomedical informatics platform, and the TRS workflow includes at least one tool register service fully qualified name (TRS_FQN). The workflow automation execution method applied in the cloud-native biomedical informatics platform includes providing a run sheet used to configure a sequencer to generate a first sequencing data, wherein the run sheet includes a sample-specific metadata associating the first sequencing data, the at least one TRS_FQN and a run identifier; importing a first data repository service (DRS) object indicating the first sequencing data in response to the run sheet, wherein a first workflow execution service fully qualified name (WES_FQN) is generated based on the run identifier and the at least one TRS_FQN and attached to the first DRS object; and performing a workflow execution service (WES) run running the TRS workflow on the first DRS object in response to the run sheet, wherein the first DRS object is resolved based on the first WES_FQN.

### BRIEF DESCRIPTION OF THE DRAWINGS

The present disclosure can be more fully understood by reading the following detailed description of the embodiment, with reference made to the accompanying drawings as follows:

FIG. 1 shows a schematic view of a workflow automation execution system according to one embodiment of the present disclosure.

FIG. 2 shows a flow chart of a workflow automation execution method applied in a cloud-native biomedical informatics platform according to one embodiment of the present disclosure.

FIG. 3 shows a schematic view of a data repository service (DRS) import flow.

FIG. 4 shows a schematic view of an example of a DRS object content.

FIG. 5 shows a schematic view of an example of a tool register service (TRS) object content.

FIG. 6 shows a schematic view of a tool register service fully qualified name (TRS_FQN) auto-matching flow.

FIG. 7A shows a flow chart of a workflow automation execution method applied in a cloud-native biomedical informatics platform according to one embodiment of the present disclosure.

FIG. 7B shows a schematic view of a first part of another example of the DRS content.

FIG. 7C shows a schematic view of a second part of the another example of the DRS content.

FIG. 7D shows a schematic view of a third part of the another example of the DRS content.

FIG. 8 shows a schematic view of a run sheet metadata.

FIG. 9A shows a schematic view of a first part of an example of an OpenWDL file that can be used in a WES run.

FIG. 9B shows a schematic view of a second part of the example of the OpenWDL file that can be used in the WES run.

FIG. 9C shows a schematic view of a third part of the example of the OpenWDL file that can be used in the WES run.

FIG. 10 shows a schematic view of another example of the TRS.

FIG. 11 shows a schematic view of runtime resolving mechanism to resolve the value of TRS_FQN based on the run sheet metadata.

FIG. 12 shows a schematic view of the TRS including FQN table, where each TRS_FQN is assigned with a cloud attribute and a local attribute.

FIG. 13 shows a schematic view of a workflow automation execution system according to one embodiment of the present disclosure.

### DETAILED DESCRIPTION

The embodiment will be described with the drawings. For clarity, some practical details will be described below. However, it should be noted that the present disclosure should not be limited by the practical details, that is, in some embodiment, the practical details is unnecessary. In addition, for simplifying the drawings, some conventional structures and elements will be simply illustrated, and repeated elements may be represented by the same labels.

It will be understood that when an element (or device) is referred to as be “connected to” another element, it can be directly connected to the other element, or it can be indirectly connected to the other element, that is, intervening elements may be present. In contrast, when an element is referred to as be “directly connected to” another element, there are no intervening elements present. In addition, the terms first, second, third, etc. are used herein to describe various elements or components, these elements or components should not be limited by these terms. Consequently, a first element or component discussed below could be termed a second element or component.

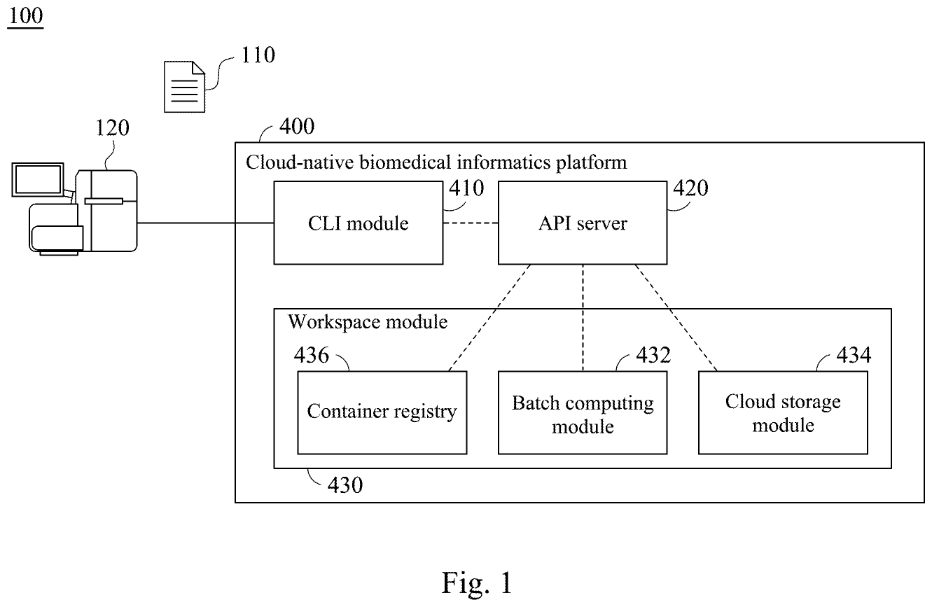

Reference is made to FIG. 1. FIG. 1 shows a schematic view of a workflow automation execution system 100 according to one embodiment of the present disclosure. The workflow automation execution system 100 includes a sequencer 120 and a cloud-native biomedical informatics platform 400. The cloud-native biomedical informatics platform 400 includes a command-line interface (CLI) module 410, an application programming interfaces (API) server 420, and a workspace module 430.

The CLI module 410 is provided as a software executable, e.g., Docker container or python package installation package source that can be installed in a data storage server connected to the sequencer 120, so that the CLI module 410 could access the sequencing data generated by the sequencer 120. The API server 420 follows Global Alliance for Genomics and Health (GA4GH) standard, and accordingly implements data repository service (DRS) API, tool register service (TRS) API, and workflow execution service (WES) API interfaces that can interact with requests and responses with the CLI module 410. The workspace module 430 provides cloud native backend resources to the cloud-native biomedical informatics platform 400. In an example, the workspace module 430 includes a batch computing module 432, a cloud storage module 434, and a container registry 436 that are respectively for providing on-demand computation, cloud storage, and container image management based on the corresponding WES, DRS, and TRS API calls triggered on the API server 420. In an example, the CLI module 410 includes corresponding Jobs, data-hub, and tools command submodules that can respectively trigger the WES, DRS, and TRS API calls to manage the backend resources of the workspace module 430.

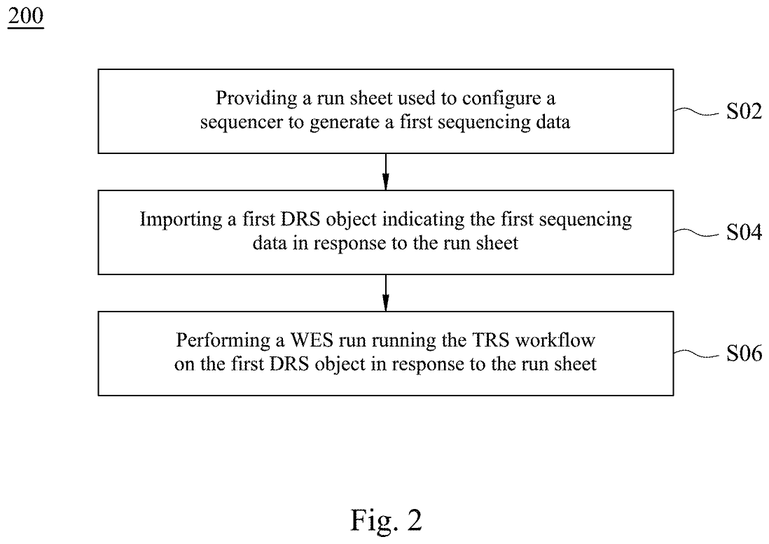

Reference is made to FIGS. 1 and 2. FIG. 2 shows a flow chart of a workflow automation execution method 200 applied in the cloud-native biomedical informatics platform 400 according to one embodiment of the present disclosure. The workflow automation execution method 200 may be applied to the workflow automation execution system 100.

The workflow automation execution method 200 with a run sheet 110 for sequencing experiments can use the run sheet 110 and the CLI module 410 to automate sequencing data processing flows. The cloud-native biomedical informatics platform 400 implements GA4GH application programming interfaces (APIs) that orchestrate industry standards, OpenWDL specifications, and cloud computing technologies, allowing clinical laboratories to manage dataset integration, workflow scalability, and regulatory compliance, and to ensure code, data, and execution integrity. The run sheet 110 contains all the mapping and configuration information about the data, workflows and workflow execution via automated content tagging. The CLI module 410 can take the run sheet as a parameter, which simplifies the entire sequencing data processing process, from taking the sequencer output to retrieving the analysis results into just a few CLI commands. In an example, the CLI module 410 further includes a run-sheet-generate submodules that can generate the run sheet 110 based on the default sample sheet defined by the sequencer 120.

In the workflow automation execution method 200, a TRS workflow is registered in the cloud-native biomedical informatics platform 400, and the TRS workflow includes at least one tool register service fully qualified name (TRS_FQN). The workflow automation execution method 200 includes a plurality of steps S02, S04, S06. The step S02 includes providing a run sheet 110 used to configure a sequencer 120 to generate a first sequencing data. For example, the first sequencing data is a piece of FASTQ data generated by the sequencer 120 using single-end sequencing technology. The run sheet 110 includes a sample-specific metadata associating the first sequencing data, the at least one TRS_FQN and a run identifier. The step S04 includes importing a first DRS object indicating the first sequencing data in response to the run sheet. A first workflow execution service fully qualified name (WES_FQN) is generated based on the run identifier and the at least one TRS_FQN and attached to the first DRS object. The step S06 includes performing a WES run running the TRS workflow on the first DRS object in response to the run sheet. The first DRS object is resolved based on the first WES_FQN. For example, the CLI module 410 includes a data-upload-runsheet and a jobs-run-runsheet commands submodules that can respectively trigger corresponding DRS, TRS, and WES APIs to execute steps S04 and S06 in response to user operations via terminal interfaces, e.g., shell.

Therefore, the workflow automation execution system 100 and the workflow automation execution method 200 of the present disclosure can simplify the entire sequencing data processing process, from taking the sequencer output to retrieving the analysis results into just a few CLI commands. In addition, the workflow automation execution system 100 and the workflow automation execution method 200 of the present disclosure can eventually transform this processing flow into a fully automated process.

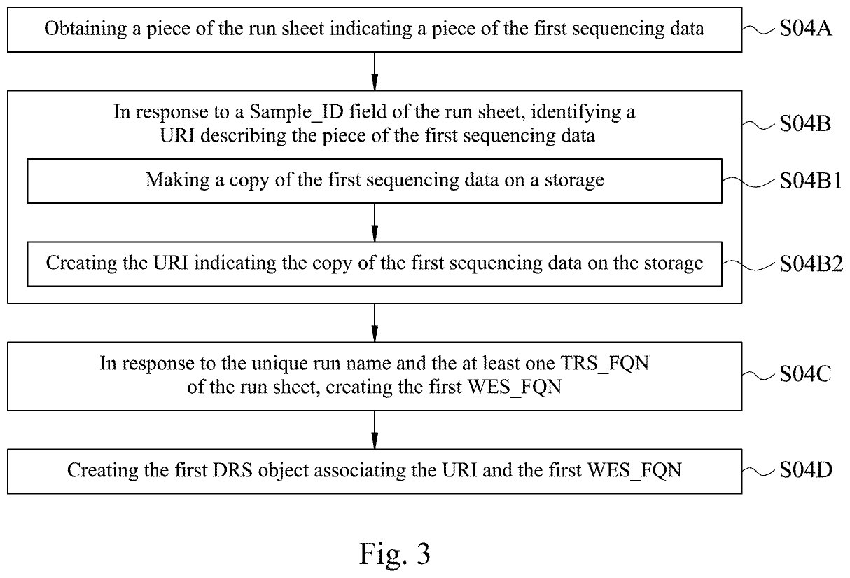

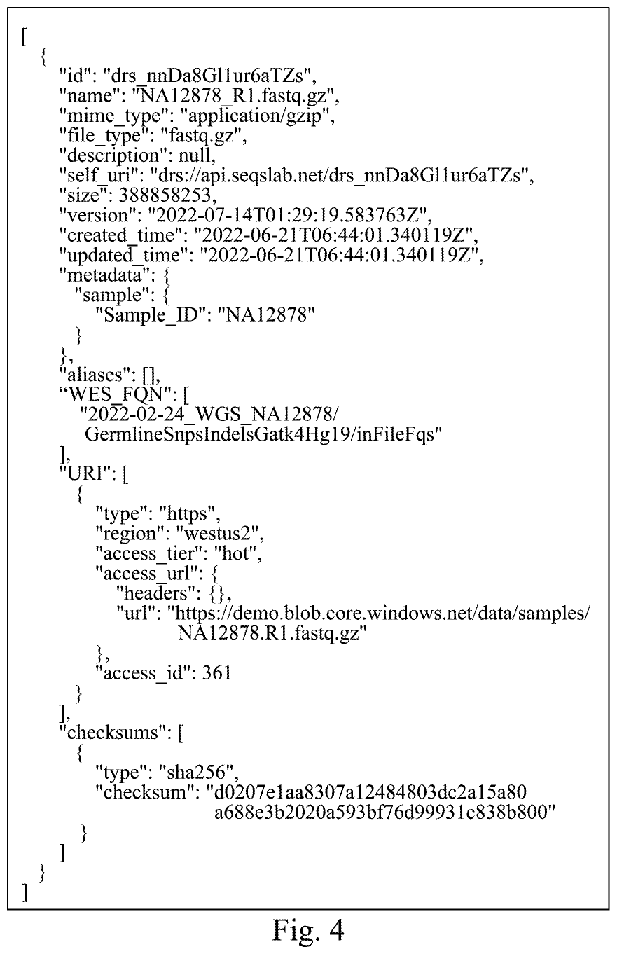

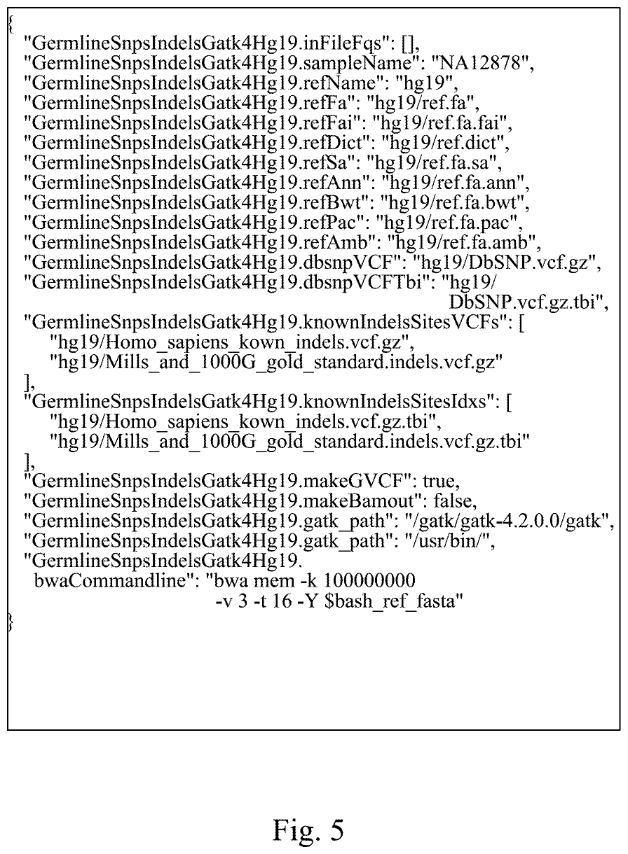

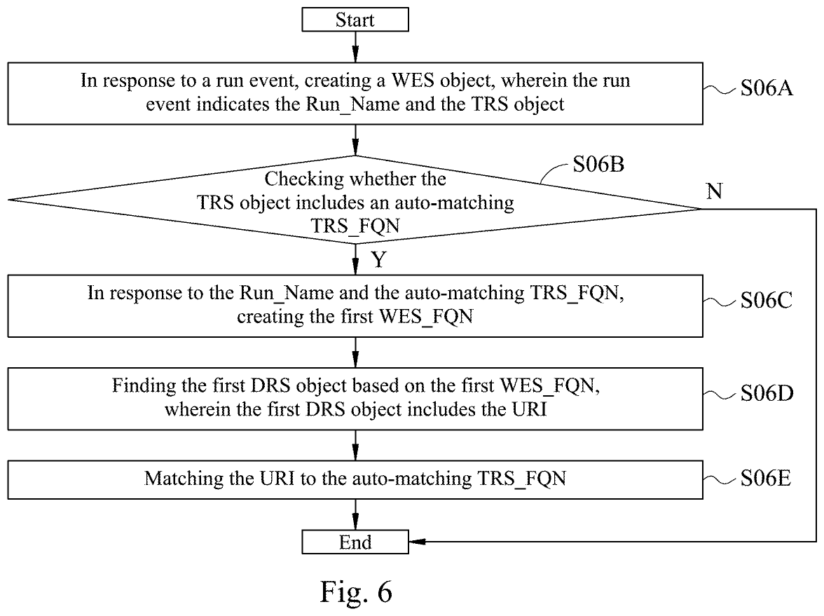

Reference is made to FIGS. 1, 2, 3, 4, 5 and 6. FIG. 3 shows a schematic view of a data repository service (DRS) import flow. FIG. 4 shows a schematic view of an example of a DRS object content. FIG. 5 shows a schematic view of an example of a tool register service (TRS) object content. FIG. 6 shows a schematic view of a TRS_FQN auto-matching flow. In an embodiment, the TRS_FQN auto-matching flow is implemented in a workflow execution service (WES) run application programming interface (API). DRS object to TRS_FQN mapping can be achieved on-the-fly in a WES run API call, so a same TRS object can be used as a template applied to multiple WES runs with the TRS_FQN corresponding to the sample DRS objects dynamically resolved.

The DRS import flow is corresponding to the step S04 of FIG. 2. The run sheet is a comma separated values (CSV) file that is directly extended from a sample sheet, a file format used by sequencer providers for storing biological sample information and metadata associated with a given experiment.

Generally, the sample sheet includes a header section and a sample section. The header section contains informational fields describing the context around which a sequencing run or analysis is performed (e.g., date, workflow, library prep kit, chemistry, etc.). The sample section includes a table of the sample-specific metadata, wherein a column of Sample_ID is defined as a unique identifier for each role of the sample-specific metadata corresponding to each of a plurality of samples submitted in the sequencer 120.

On the basis of the sample sheet, the run sheet further defines five additional columns (sample-specific metadata) for each row of the sample section. The sample-specific metadata includes a DRS identification data DRS_ID, a first additional data Read1_TRS_FQN, a run name data Run_Name, a workflow uniform resource locator data Workflow URL and a runtime data Runtimes. The run sheet includes the header section and the sample section. The run sheet serves as a dry lab overview plan and is configured to specify mapping among the sequencing data, the DRS object, the TRS workflow and the WES run for all samples submitted in the sequencer 120, so that the sequencing data, the DRS object, the TRS workflow and the WES run are transformed into a fully automated process by a processor. The run sheet is also a critical input for the CLI module 410 in the dry lab daily routine, serving as a link for the entire process from sample FASTQ uploading, DRS registration, DRS objects to TRS workflows mapping, and eventually to WES execution. For example, the process of sample FASTQ uploading and DRS registration is shown in FIG. 3, and the corresponding DRS content is shown in FIG. 4.

In FIGS. 2 and 3, the workflow automation execution method 200 further includes a plurality of steps S04A, S04B, S04C, S04D. The step S04A includes obtaining a piece of the run sheet indicating a piece of the first sequencing data. The step S04B includes in response to a sample identification (Sample_ID) field of the run sheet, identifying a uniform resource identifier (URI) describing the piece of the first sequencing data. For example, the URI could be file names indicating the physical copy of the first sequencing data on a server. The step S04C includes in response to the unique run name and the at least one TRS_FQN of the run sheet, creating the first WES_FQN. The step S04D includes creating the first DRS object associating the URI and the first WES_FQN.

In another example, the step S04B further includes a plurality of steps S04B1, S04B2. The step S04B1 includes making a copy of the first sequencing data on a storage. The step S04B2 includes creating the URI indicating the copy of the first sequencing data on the storage. For example, the storage could be a cloud storage, e.g., Azure storage account, and step S04B1 and S04B2 are achieved by uploading the first sequencing data onto the cloud storage and obtaining the URI indicating the data stored in the cloud storage.

The DRS identification data DRS_ID associates a physical sample file to the first DRS object by assigning a DRS_ID rule using the sample-specific metadata. The first DRS object may be a data-virtualized DRS object. In detail, the CLI module 410 uses the sample-sheet package to do run sheet parsing and generate a run sheet metadata for each individual sequenced FASTQ file during the samples upload and registration processes. In an example, the run sheet metadata corresponding to a pair-1 FQSTQ file NA12878_R1.fastq.gz includes a header section, a sample section and a file section. The header section and the sample section respectively include information depicted in the header section and the sample section of the sample sheet, and the file section indicates the pairing information of the pair-1 FASTQ file. The DRS_ID rule can use a nested dictionary syntax that is chained with a hyphen (-) as a separator character. For example, {header. Date}-{sam ple. Description}-{sam ple.Sample_ID}-{file.Pair}, which will render the DRS_ID as 20220224-WGS-NA12878-1 for the sample FASTQ file NA12878_R1.fastq.gz.

The first additional data Read1_TRS_FQN indicates how to associate the first DRS object to a first element of the at least one TRS_FQN. In the clinical genetic testing operation context, the dry-lab workflow of a sequencing sample is determined before the sequencing operation, and the dry-lab workflow will be registered as a TRS workflow object. The dry-lab workflow is described in Workflow Description Language (WDL) protocol and the registered TRS workflow object includes an inputs.json shown in FIG. 5. For example, the TRS_FQN GermlineSnpsIndelsGatk4Hg19.inFileFqs indicates the FASTQ sequencing files required by the dry-lab workflow, and the first additional data Read1_TRS_FQN GermlineSnpsIndelsGatk4Hg19.inFileFqs indicates that a read1 DRS object (i.e., the first DRS object) should be associated to the first element of the TRS_FQN GermlineSnpsIndelsGatk4Hg19.inFileFqs. In addition, the first WES_FQN is generated based on the run identifier and the first additional data Read1_TRS_FQN and attached to the first DRS object, thereby establishing mapping among the first sequencing data, the first DRS object and the at least one TRS_FQN.

The run name data Run_Name associates the first DRS object to a specific WES run by assigning a unique and indicative run name, generally based on the run sheet, e.g., 2022-02-24_WGS_NA12878. In detail, the run name data Run_Name specifies a unique name for a given WES run. The run name data Run_Name is created based on the run sheet metadata to make it both unique and meaningful. For a multi-sample WES run use case, the template {header.Date}_{sample.Description} is used for the run name data Run_Name to render a run name data Run_Name 2022-02-24_WGS shared by multiple WGS samples in a sequencing run from 2022-02-24. For a single-sample WES run use case, the template {header.Date}_{sample.Description}_{sample.Sample_ID} is used to render a sample specific run name data Run_Name 2022-02-24_WGS_NA12878 for a specific WGS sample NA12878 in a sequencing run from 2022-02-24.

The CLI module 410 creates the first WES_FQN based on the run name data Run_Name and the first additional data Read1_TRS_FQN to designate a unique association among a WES run to the corresponding DRS object. In an example, the first WES_FQN 2022-02-24 WGS NA12878/GermlineSnpsIndelsGatk4Hg19/inFileFqs is created and attached to the first DRS object 20220224-WGS-NA12878-1, wherein the character ‘.’ in the first additional data Read1_TRS_FQN is replaced with 7′ during the construction of the first WES_FQN.

The workflow uniform resource locator data Workflow_URL (TRS workflow_url) specifies a TRS object to be used for the WES run. An inputs.json example of the TRS is shown in FIG. 5, where the TRS_FQN GermlineSnpslndelsGatk4Hg19.inFileFqs is empty, indicating that it is an auto-matching TRS_FQN, as shown in FIG. 6. Workflow_URL specifies a TRS object that is going to be applied on the given sample.

The runtime data Runtimes specifies a WES execution runtimes configuration in a format of key-value pairs of WDL call-name and WES runtime options, e.g., GermlineSnpsIndelsGatk4Hg19=acu-m641:BwaMem=acu-m4805. The WDL call-name indicates a specific workflow stage of the TRS workflow, and the WES runtime options indicates a runtime environment specification, e.g., how many nodes in what virtual machine (VM) size to be used. For example, acu-m641 indicates a cluster specification of 3 dedicated and 5 low-priority Azure Standard_D13_v2 instances, and acu-m480s indicates a cluster specification of 20 dedicated and 10 low-priority Azure Standard_D14_v2 instances.

Given the above, the run sheet includes original columns of the sample sheet and the five additional columns for one row of the sample section. Table 1 lists Header and Reads. Table 2.1 lists the original columns of the sample sheet for one row of the sample section. Tables 2.2 and 2.3 list the five additional columns for one row of the sample section. Tables 1, 2.1, 2.2 and 2.3 are combined to form the run sheet, as seen in the following example:

TABLE 1
[Header]

| | |
|---|---|
| Date | Feb. 24, 2022 |
| Application | NextSeq FASTQ Only |
| Instrument Type | NovaSeq6000 |
| Assay | Illumina WGS |
| Index Adapters | IDT-ILMN DNA-RNA UDP Indexes |
| Chemistry | N/A |

[Reads]

| | |
|---|---|
| 151 | N/A |
| 151 | N/A |

TABLE 2.1
[Data]

| | Sample_ | | | | |
|---|---|---|---|---|---|
| Sample_ID | Type | Index_ID | Index | Index2 | Description |
| NA12878 | Patient- | UDP0024 | CGTACT | AGAGGA | WGS |
| | Sample | | AG | TA | |

TABLE 2.2
[Data]

| DRS_ID | Read1_TRS_FQN | Run_Name |
|---|---|---|
| {header.Date}- | GermlineSnpsIndelsGatk4Hg19.inFileFqs | 2022-02- |
| {sample.Description}- | | 24_WGS_NA12878 |
| {sample.Sample_ID}- | | |
| {file.Pair} | | |

TABLE 2.3
[Data]

| Workflow_URL | Runtimes |
|---|---|
| https://api.seqslab.net/trs/v2/tools/ | GermlineSnpsIndelsGatk4Hg19=acu- |
| trs_G3A9QuumbKxuSvl/ | m64l:BwaMem=acu-m480s |
| versions/1.0/WDL/files/ | |

In FIGS. 2 and 6, the workflow automation execution method 200 further includes a plurality of steps S06A, S06B, S06C, S06D, S06E. The step S06A includes in response to a run event, creating a WES object, wherein the run event indicates the run name data (Run_Name) and the TRS object. The step S06B includes checking whether the TRS object includes an auto-matching TRS_FQN. In an embodiment, the step S06B determines whether a given TRS_FQN is auto-matching based on its corresponding value is empty array in the inputs.json. In the example shown in FIG. 5, the TRS_FQN GermlineSnpindelsGatk4Hg19.inFileFqs corresponds to empty array, and the TRS_FQN is determined auto-matching. On the contrary, the TRS_FQN GermlineSnpindelsGatk4Hg19.refFa does not correspond to empty array, and the TRS_FQN is determined as not auto-matching. Though only the embodiment of indicating an auto-matching TRS_FQN using the value of empty array in the inputs.json, the invention is not limited thereto. In other embodiment, TRS_FQN auto-matching may also be determined based on other forms of legitimate json values indicating empty values, e.g., empty string or null, in the inputs.json or other possible data structure kept in the TRS object.

If the result of the step S06B is “Y”, the steps S06C, S06D, S06E are performed. If the result of the step S06B is “N”, the steps S06C, S06D, S06E are not performed. The step S06C includes in response to the run name data (Run_Name) and the auto-matching TRS_FQN, creating the first WES_FQN. The step S06D includes finding the first DRS object based on the first WES_FQN, wherein the first DRS object includes a uniform resource identifier (URI). The step S06E includes matching the URI to the auto-matching TRS_FQN. The auto-matching TRS_FQN represents that a content of a column of the TRS object is empty.

In an example, the DRS service supports DRS object query based on the WES_FQN, so that DRS run-time resolving can be achieved based on the run name data Run_Name and the TRS_FQN, as shown in FIG. 6. In an embodiment, the flow in FIG. 6 is implemented in WES run API.

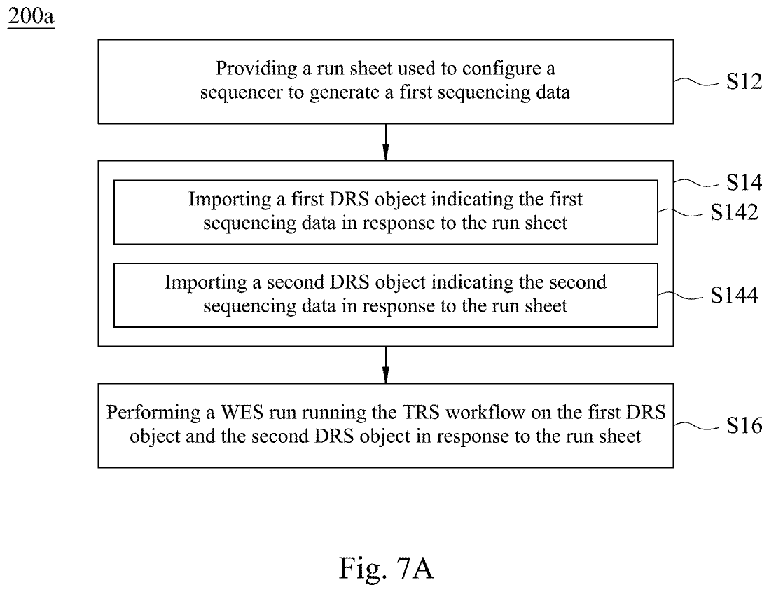

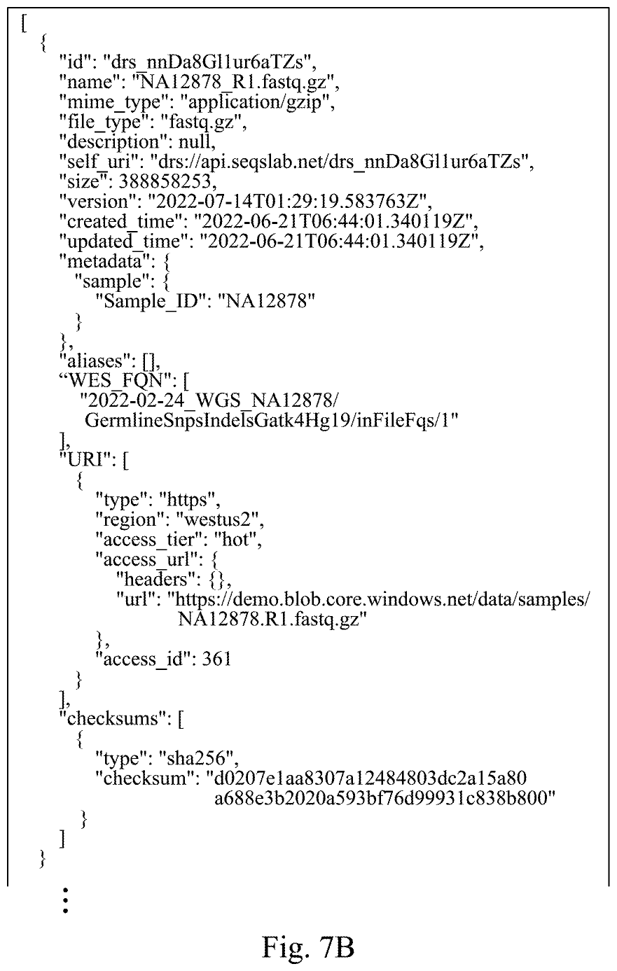

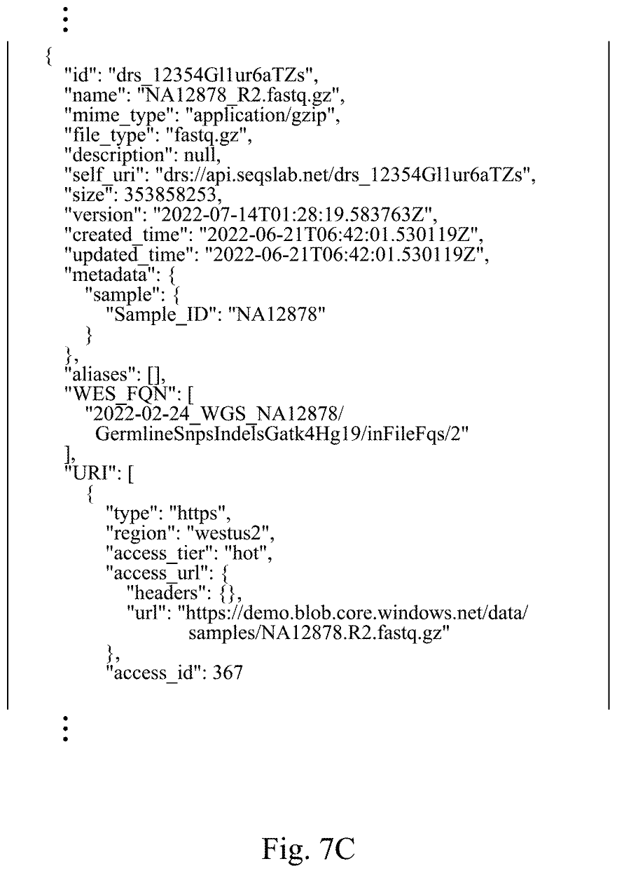

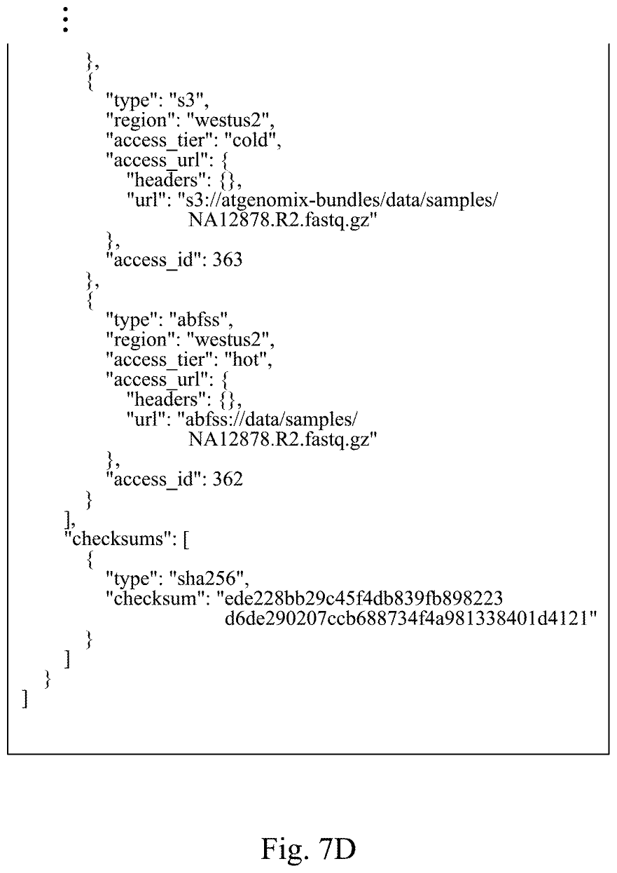

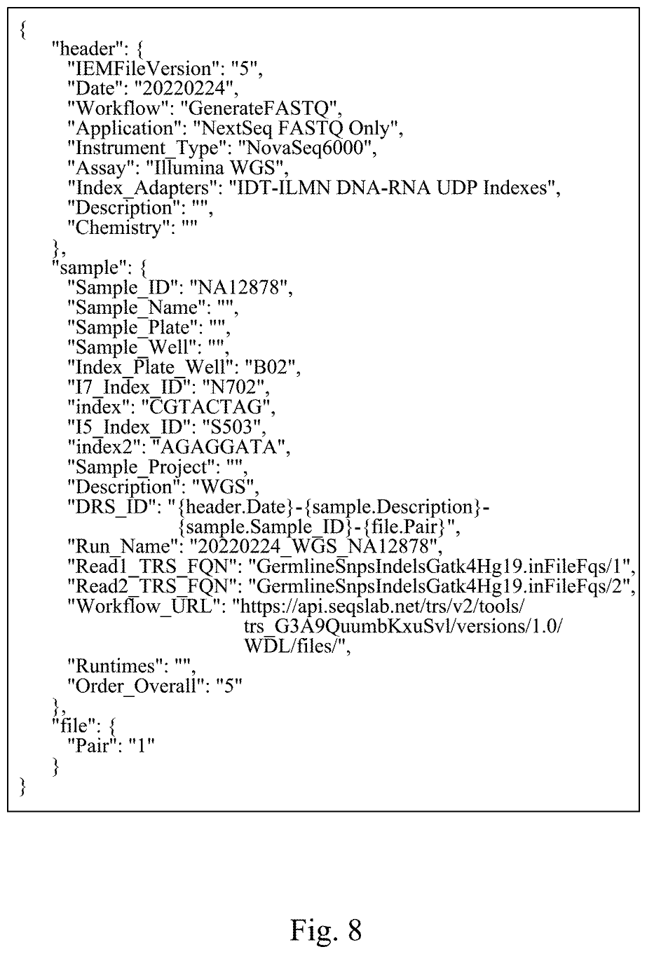

Though only the example that the sequencer 120 generates the first sequencing data, and building the association among the first sequencing data, the DRS object, and the WES run with steps S02 to S06 are illustrated in the present embodiment of the invention, the invention is not limited thereto. In other example, the sequencer 120 can also generate more than one sequencing data, and the same mechanism can be employed to build the association among those sequencing data, the DRS objects, and the WES run. For example, the sequencer 120 is configured to conduct paired-end sequencing and accordingly generate two pieces of sequencing data respectively corresponding to the first and the second pair of FASTQ files. Reference is made to FIGS. 1, 7A, 7B, 7C, 7D and 8. FIG. 7A shows a flow chart of a workflow automation execution method 200a applied in a cloud-native biomedical informatics platform 400 according to one embodiment of the present disclosure. FIG. 7B shows a schematic view of a first part of another example of the DRS content. FIG. 7C shows a schematic view of a second part of the another example of the DRS content. FIG. 7D shows a schematic view of a third part of the another example of the DRS content. FIG. 8 shows a schematic view of a run sheet metadata. The workflow automation execution method 200a may be applied to the workflow automation execution system 100 of FIG. 1.

The workflow automation execution method 200a includes a plurality of steps S12, S14, S16. The step S12 includes providing a run sheet 110 used to configure a sequencer 120 to generate a sequencing data set. The sequencing data set includes a first sequencing data and a second sequencing data. The run sheet 110 includes a sample-specific metadata associating the sequencing data set, the at least one TRS_FQN and a run identifier. The step S14 includes a plurality of steps S142, S144. The step S142 includes importing a first DRS object indicating the first sequencing data in response to the run sheet 110. A first WES_FQN is generated based on the run identifier and the at least one TRS_FQN and attached to the first DRS object. The step S144 includes importing a second DRS object indicating the second sequencing data in response to the run sheet 110. A second WES_FQN is generated based on the run identifier and the at least one TRS_FQN and attached to the second DRS object. The step S16 includes performing a WES run running the TRS workflow on the first DRS object and the second DRS object in response to the run sheet 110. The first DRS object and the second DRS object are resolved based on the first WES_FQN and the second WES_FQN, respectively.

On the basis of the sample sheet, the run sheet 110 further defines six additional columns (sample-specific metadata) for each row of the sample section. The sample-specific metadata includes a DRS identification data DRS_ID, a first additional data Read1_TRS_FQN, a second additional data Read2_TRS_FQN, a run name data Run_Name, a workflow uniform resource locator data Workflow URL and a runtime data Runtimes. Tables 2.2A and 2.3 list the six additional columns for one row of the sample section.

TABLE 2.2A
[Data]

| DRS_ID | Read1_TRS_FQN | Read2_TRS_FQN | Run_Name |
|---|---|---|---|
| {header.Date}- | GermlineSnpsIndelsGatk4Hg19.inFileFqs/1 | GermlineSnpsIndelsGatk4Hg19.inFileFqs/2 | 2022-02- |
| {sample.Description}- | | | 24_WGS_NA12878 |
| {sample.Sample_ID}- | | | |
| {file.Pair} | | | |

The DRS identification data DRS_ID associates physical sample files to the first DRS object and the second DRS object by assigning a DRS_ID rule using the sample-specific metadata. Each of the first DRS object and the second DRS object may be a data-virtualized DRS object. In detail, the CLI module 410 uses the sample-sheet package to do run sheet parsing, and generates a run sheet metadata for each individual sequenced FASTQ file during the samples upload and registration processes. In an example, the run sheet metadata corresponding to a pair-1 FQSTQ file NA12878_R1.fastq.gz includes a header section, a sample section and a file section, as shown in FIG. 8. The header section and the sample section respectively include information depicted in the header section and the sample section of the sample sheet, and the file section indicates the pairing information of the pair-1 FASTQ file. The DRS_ID rule can use a nested dictionary syntax that is chained with a hyphen (-) as a separator character. For example, {header.Date}-{sample.Description}-{sample.Sample_ID}-{file.Pair}, which will render the DRS identification data DRS_ID as 20220224-WGS-NA12878-1 for the sample FASTQ file NA12878 R1.fastq.gz. Similarly, for paired-end sequencing scenario, the DRS identification data DRS_ID as 20220224-WGS-NA12878-2 for the sample FASTQ file NA12878_R2.fastq.gz is rendered.

The first additional data Read1_TRS_FQN indicates how to associate the first DRS object to a first element of the at least one TRS_FQN. In the clinical genetic testing operation context, the dry-lab workflow of a sequencing sample is determined before the sequencing operation, and the dry-lab workflow will be registered as a TRS workflow object. In an example, the dry-lab workflow is described in Workflow Description Language (WDL) protocol and the registered TRS workflow object includes an inputs.json shown in FIG. 5, wherein the inputs.json includes a plurality of TRS_FQN to DRS_URI key-value pairs. For example, the TRS_FQN GermlineSnpslndelsGatk4Hg19.inFileFqs indicates the FASTQ sequencing files required by the dry-lab workflow, and the first additional data Read 1_TRS_FQN GermlineSnpslndelsGatk4Hg19.inFileFqs/1 indicates that a read1 DRS object (i.e., the first DRS object) should be associated to the first element of the TRS_FQN GermlineSnpsIndelsGatk4Hg19.inFileFqs. In addition, the first WES_FQN is generated based on the run identifier and the first additional data Read1_TRS_FQN and attached to the first DRS object, thereby establishing mapping among the first sequencing data, the first DRS object and the at least one TRS_FQN. The second WES_FQN is generated based on the run identifier and the second additional data Read2 TRS_FQN and attached to the second DRS object, thereby establishing mapping among the second sequencing data, the second DRS object and the at least one TRS_FQN.

The second additional data Read2_TRS_FQN indicates how to associate the second DRS object to a second element of the at least one TRS_FQN. Similar to the first additional data Read1_TRS_FQN, for paired-end sequenced samples, the second additional data Read2_TRS_FQN of GermlineSnpslndelsGatk4Hg19.inFileFqs/2 indicates that a read2 DRS object (i.e., the second DRS object) should be associated to the second element of the TRS_FQN GermlineSnpsIndelsGatk4Hg19.inFileFqs.

With the addition of the DRS identification data DRS_ID, the first additional data Read1_TRS_FQN, and the second additional data Read2_TRS_FQN, the run sheet 110 establishes the association among the sequenced FASTQ files, the virtualized DRS objects, and the TRS_FQN of a TRS object, in a both human-readable and machine-recognizable manner.

The run name data Run_Name associates the first DRS object and the second DRS object to the WES run by assigning a unique run name based on the run sheet 110. The workflow uniform resource locator data Workflow URL specifies a TRS object to be used for the WES run. The runtime data Runtimes specifies a WES execution runtimes configuration in a format of key-value pairs of WDL call-name and WES runtime options.

The CLI module 410 creates the first WES_FQN and the second WES_FQN based on the run name data Run_Name, the first additional data Read1_TRS_FQN and the second additional data Read2 TRS_FQN to designate a unique association among a WES run to the corresponding DRS objects. In an example, the first WES_FQN 2022-02-24 WGS NA12878/GermlineSnpsIndelsGatk4Hg19/inFileFqs/1 is created and attached to the first DRS object 20220224-WGS-NA12878-1, and the second WES_FQN 2022-02-24 WGS NA12878/GermlineSnpsIndelsGatk4Hg19/inFileFqs/2 is created attached to the second DRS object 20220224-WGS-NA12878-2, wherein the character ‘.’ in the first additional data Read1_TRS_FQN and the second additional data Read2 TRS_FQN are replaced with “l” during the construction of WES_FQNs.

In an example, the WES_FQN based DRS object query supports a directory-like, hierarchical query, so that the run name data Run_Name itself can be used as a root query condition to find all DRS objects associated with the given run name data Run_Name.

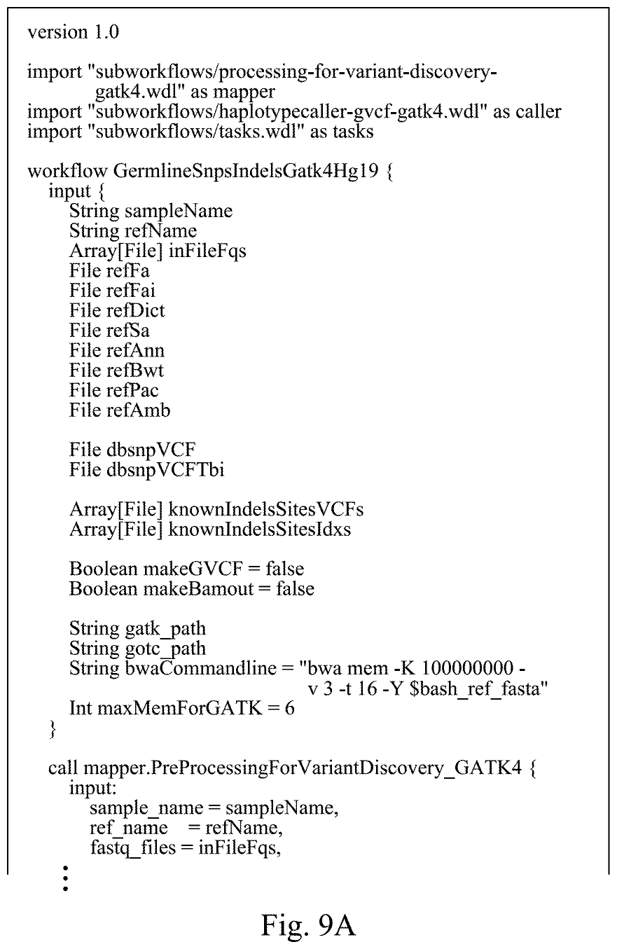

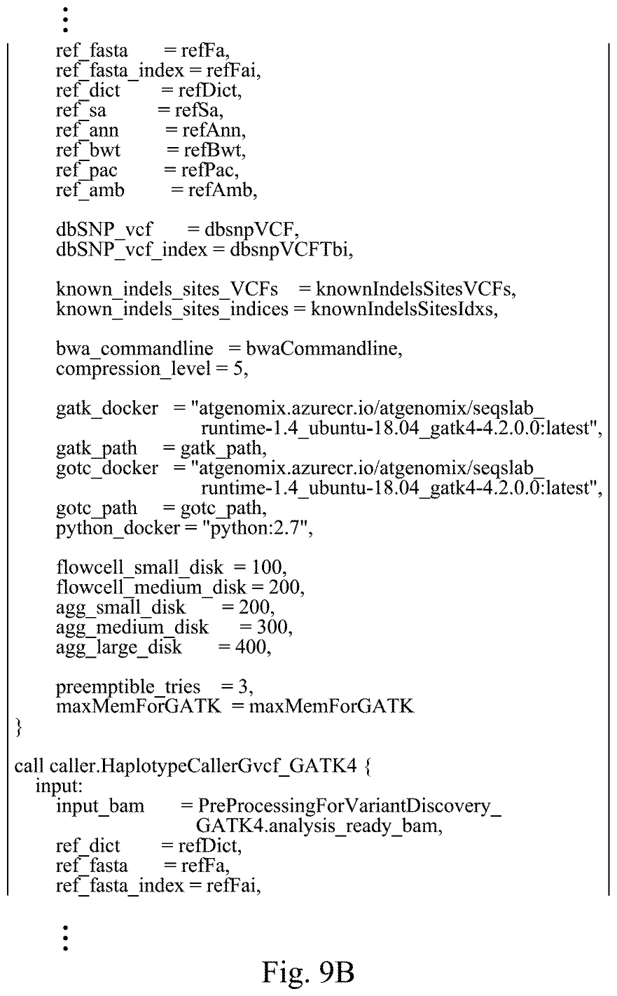

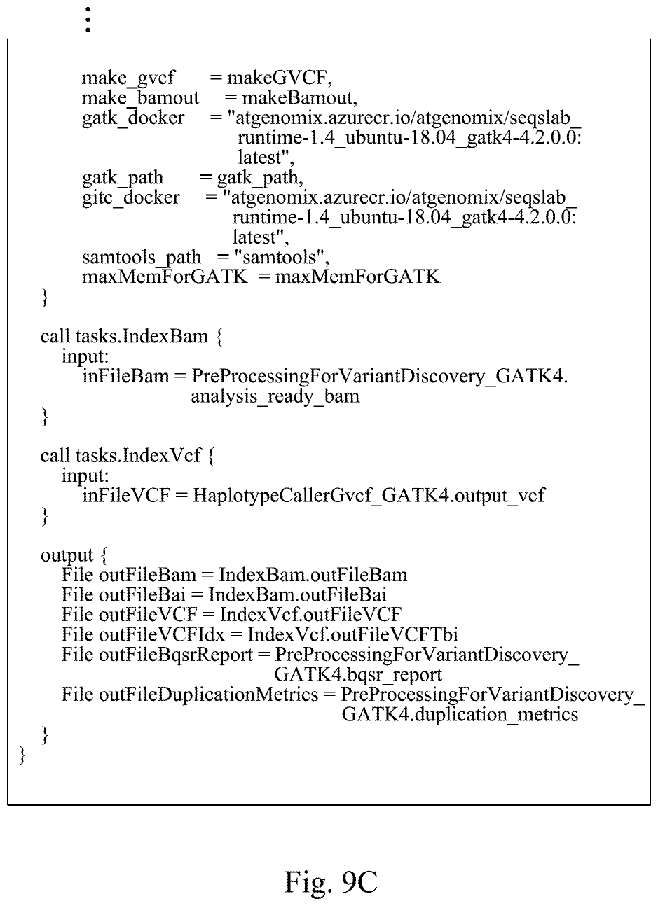

Reference is made to FIGS. 9A, 9B and 9C. FIG. 9A shows a schematic view of a first part of an example of an OpenWDL file that can be used in a WES run. FIG. 9B shows a schematic view of a second part of the example of the OpenWDL file that can be used in the WES run. FIG. 9C shows a schematic view of a third part of the example of the OpenWDL file that can be used in the WES run. The output files generated by the WES run are also registered with corresponding WES_FQNs, e.g., 2022-02-24 WGS NA12878/GermlineSnpsIndelsGatk4Hg19/outFileBam and 2022-02-24 WGS NA12878/GermlineSnpsIndelsGatk4Hg19/outFileVCF to enhance future data accessibility, where FQNs outFileBam and outFileVCF are recited in the output section of the workflow GermlineSnnpslnndelsGatk4Hg19.

For example, the TRS_FQN GermlineSnpslndelsGatk4Hg19.refFa indicates the reference genome FASTA file required by the dry-lab workflow, and the DRS_URI hg19/ref.fa indicates the file path of the reference genome FASTA file. Though only the example that the DRS_URI indicates the file path is shown here, the DRS_URI is not limited thereto, and can also indicate other logical or physical resources, e.g., DRS object ID, DRS object WES_FQN attribute, DRS object tag attribute or cloud storage access method.

The user can use the CLI module to upload either individual files or entire directories to the Data Hub using a datahub upload command. Alternatively, the user can use the run sheet to upload sample FASTQ files by preparing the run sheet file and then running the datahub upload-runsheet command. Doing so outputs the upload.json in the stdout of the command process. In an example, the datahub upload-runsheet command takes the input of an input-directory containing the FASTQ files, and the run-sheet file reciting all the metadata for those FASTQ files, as listed in Table 3.

TABLE 3

```
seqslab datahub upload-runsheet \
  --run-sheet /home/run-2022-02-26.csv \
  --input-dir /volume/fastq/2022-02-14/ \
  --workspace seqslabwus2 > upload.json
```

Cloud-native biomedical informatics platforms in the cloud with an Azure backend use the Azure Block List API whenever the user runs the CLI datahub upload command. This enables files to be programmatically broken up into blocks, uploaded in parallel, and re-assembled in the cloud storage as a block blob. As such, even if the datahub upload command is executed multiple times, all successfully uploaded blocks are kept in the Azure cloud storage as cache and only the failed blocks will be re-transmitted, resulting to a highly efficient and fault-resilient data transmission.

Running the datahub upload-runsheet command provides an upload.json object for each uploaded sample file. For example, the upload.json has the content shown in FIG. 4. Apart from automatically populating the storage related fields, the metadata fields are also filled out based on the sample sheet information that was extracted from the run sheet, as shown in FIG. 8.

After the sample files are uploaded, the user can then use the datahub register command to complete the DRS registration process based on the upload.json, as listed in Table 4.

TABLE 4

```
seqslab datahub register \
  file-blob \
  --workspace seqslabwus2 \
  --stdin < upload.json > register.json
```

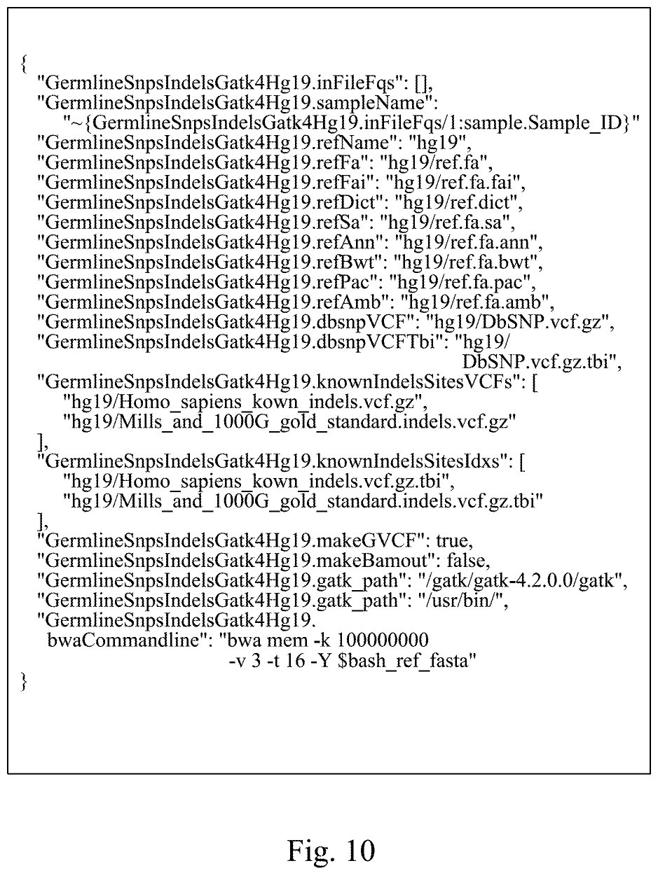

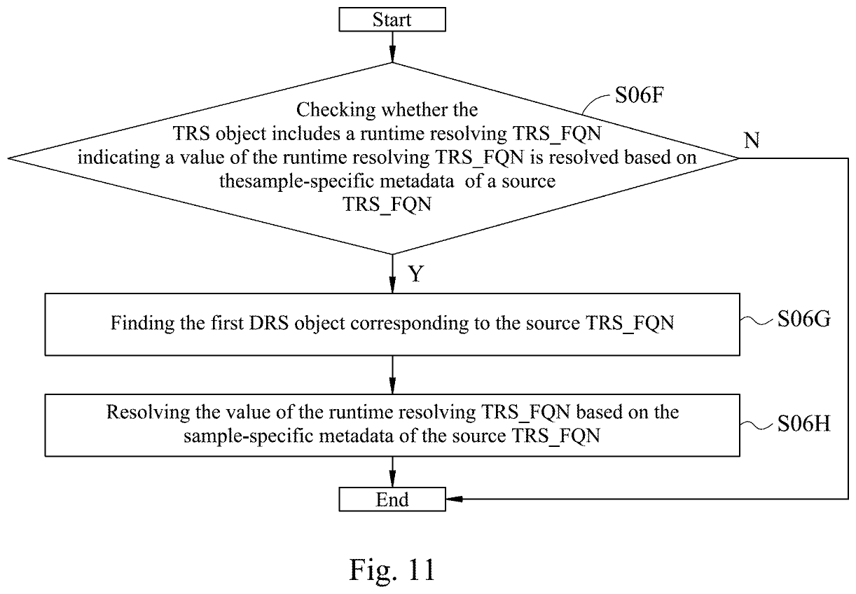

Reference is made to FIGS. 5, 6, 8, 10 and 11. FIG. 10 shows a schematic view of another example of the TRS. FIG. 11 shows a schematic view of runtime resolving mechanism to resolve the value of tool register service fully qualified name (TRS_FQN) based on the run sheet metadata. Though only the situation that the TRS includes inputs.json shown in FIG. 5 is illustrated, the TRS is not limited thereto. In other example, the inputs.json could also include runtime resolving TRS_FQN, wherein its value is resolved based on the run sheet metadata of the DRS object assigned to other TRS_FQN. The inputs.json shown in FIG. 10 includes a TRS_FQN GermlineSnpsIndelsGatk4Hg19.sampleName configured as “-{GermlineSnpsIndelsGatk4Hg19.inFileFqs/1:sample.Sample_ID}”, indicating that the value of this TRS_FQN GermlineSnpslndelsGatk4Hg19.sampleName will be dynamic resolved based on the run sheet metadata sample.Sample_ID of the DRS object matched to the TRS_FQN GermlineSnpslndelsGatk4Hg19.inFileFqs/1, e.g., the string value of “NA12878” based on the example shown in FIG. 8. Besides, the method of FIG. 6 further includes a plurality of steps S06F, S06G, S06H shown in FIG. 11 to resolve the value of TRS_FQN based on the run sheet metadata. Though only the format of “-{FQN:run sheet metadata}” format is exemplified as an example in the present disclosure, the embodiment is not limited thereto, and could apply other template formats to indicate the mapping of the source FQN and the run sheet metadata.

Though only the situation that the DRS metadata having the format of nested json shown in FIG. 8, and the dynamic resolving expression as “-{GermlineSnpsIndelsGatk4Hg19.inFileFqs/1:sample.Sample_ID}” is illustrated as an example in the present embodiment, the invention is not limited thereto. In other example, the DRS metadata can also be in other legitimate json format, and the other possible dynamic resolving expression, e.g., jsonpath-based expression can be used to retrieving components from the metadata.

In FIG. 11, the step S06F includes checking whether the TRS object includes a runtime resolving TRS_FQN indicating a value of the runtime resolving TRS_FQN is resolved based on the sample-specific metadata of a source TRS_FQN. If the result of the step S06F is “Y”, the steps S06G, S06H are performed. If the result of the step S06F is “N”, the steps S06G, S06H are not performed. The step S06G includes finding the first DRS object corresponding to the source TRS_FQN. The step S06H includes resolving the value of the runtime resolving TRS_FQN based on the sample-specific metadata of the source TRS_FQN.

Though only the example that the run sheet of the content in Tables 1, 2.1, 2.2 and 2.3, and the corresponding run sheet metadata of the content in FIG. 8 is illustrated, the embodiment is not limited thereto. In other example, the run sheet can also be extended to attach more sample related information fields, e.g., ISO 4454:2022 phenopacket record, personal health records, electronic medical records, electronic health records or family disease history. Those extended fields in the run sheet will also be extracted and added to the run sheet metadata of the corresponding DRS object. As such, through the runtime resolving mechanism shown in FIG. 11, those extended sample related fields can be applied to any of the TRS_FQNs for downstream analysis.

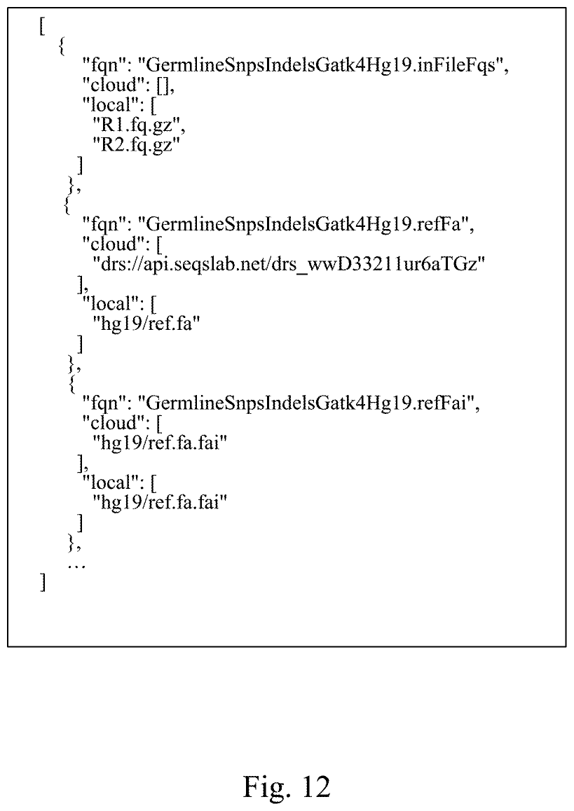

Reference is made to FIGS. 5, 6 and 12. FIG. 12 shows a schematic view of the TRS including FQN table, where each TRS_FQN is assigned with a cloud attribute and a local attribute. Though only the situation that the TRS includes inputs.json shown in FIG. 5 is illustrated, the embodiment is not limited thereto. In other example, the TRS may include FQN table shown in FIG. 12, where each TRS_FQN is assigned with a cloud attribute and a local attribute, respectively indicating a generic DRS URI or DRS label, and a local URI of the DRS object. Whether the TRS_FQN is an auto-matching TRS_FQN is indicated by whether the cloud attribute is empty; if so, as depicted by the TRS_FQN GermlineSnpslnndelsGatk4Hg19.inFileFqs, the flow of FIG. 6 will be proceeded.

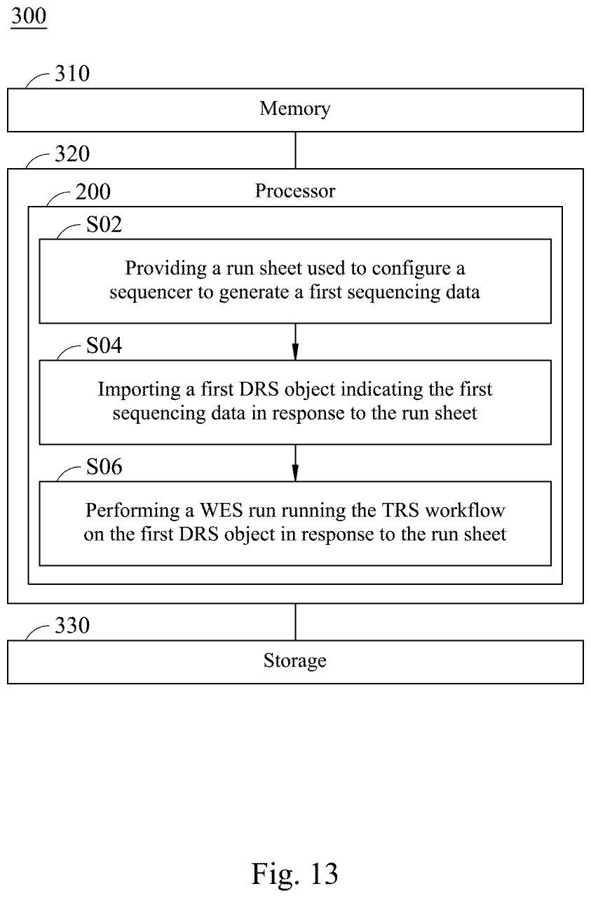

Reference is made to FIGS. 2, 7A and 13. FIG. 13 shows a schematic view of a workflow automation execution system 300 according to one embodiment of the present disclosure. The workflow automation execution methods 200, 200a of FIGS. 2, 7A may be applied to the workflow automation execution system 300.

The workflow automation execution system 300 includes a cloud-native biomedical informatics platform. The cloud-native biomedical informatics platform includes a memory 310, a processor 320 and a storage 330. The memory 310 stores a run sheet, a first sequencing data and a second sequencing data. The processor 320 is signally connected to the memory 310 and configured to perform one of the workflow automation execution methods 200, 200a (FIG. 13 only shows the workflow automation execution method 200). The storage 330 is signally connected to the processor 320 and stores a copy of the first sequencing data or the second sequencing data. The memory 310 may include a random access memory (RAM) or another type of dynamic storage device that may store information and instructions for execution by the processor 320. The processor 320 may include any type of processor, microprocessor, cloud processor or GPU. The processor 320 may include a single device (e.g., a single core) and/or a group of devices (e.g., multi-cores). The storage 330 may be any type of dynamic or static storage device that may store information.

It is understood that one of the workflow automation execution methods 200, 200a of the present disclosure is performed by the aforementioned steps. A computer program of the present disclosure stored on a computer readable medium is used to perform the method described above. The aforementioned embodiments can be provided as a computer program product, which may include the computer readable medium on which instructions are stored for programming a computer (or other electronic devices) to perform a process based on the embodiments of the present disclosure. The computer readable medium can be, but is not limited to, a floppy diskette, an optical disk, a compact disk-read-only memory (CD-ROM), a magneto-optical disk, a read-only memory (ROM), a random access memory (RAM), an erasable programmable read-only memory (EPROM), an electrically erasable programmable read-only memory (EEPROM), a magnetic or optical card, a flash memory, or another type of media/computer readable medium suitable for storing electronic instructions. Moreover, the embodiments of the present disclosure also can be downloaded as a computer program product, which may be transferred from a remote computer to a requesting computer by using data signals via a communication link (such as a network connection or the like).

In addition, the workflow automation execution systems 100, 300, the workflow automation execution methods 200, 200a and the computer readable medium of the present disclosure can use a technique for automating multimodal computational workflows. The technique for automating multimodal computational workflows can realize the automation and optimization of processing biomedical data by unifying WDL, common workflow language (CWL), YAML, XML, structured query language (SQL) and machine learning (ML) workflows with in-memory computing optimization. Moreover, the technique for automating multimodal computational workflows can support other analysis tools for multimodal, such as Python, R, etc. In other words, the data parallelism combined with the GPU accelerators may be performed via a plurality of steps of an operator pipeline.

According to the aforementioned embodiments and examples, the advantages of the present disclosure are described as follows.
1. The present disclosure can simplify the entire sequencing data processing process, from taking the sequencer output to retrieving the analysis results into just a few CLI commands.
2. The present disclosure can eventually transform this processing flow into a fully automated process.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A workflow automation execution method applied in a cloud-native biomedical informatics platform, wherein a tool register service (TRS) workflow is registered in the cloud-native biomedical informatics platform, and the TRS workflow comprises at least one tool register service fully qualified name (TRS_FQN), the workflow automation execution method applied in the cloud-native biomedical informatics platform comprising:
providing a run sheet used to configure a sequencer to generate a first sequencing data, wherein the run sheet comprises a sample-specific metadata associating the first sequencing data, the at least one TRS_FQN and a run identifier;
importing a first data repository service (DRS) object indicating the first sequencing data in response to the run sheet, wherein a first workflow execution service fully qualified name (WES_FQN) is generated based on the run identifier and the at least one TRS_FQN and attached to the first DRS object; and
performing a workflow execution service (WES) run running the TRS workflow on the first DRS object in response to the run sheet, wherein the first DRS object is resolved based on the first WES_FQN.

2. The workflow automation execution method applied in the cloud-native biomedical informatics platform of claim 1, wherein the run sheet is a comma separated values (CSV) file and further comprises:
a header section comprising informational fields describing a context around which a sequencing run or analysis is performed; and
a sample section comprising the sample-specific metadata corresponding to each of a plurality of samples submitted in the sequencer;
wherein the run sheet is configured to specify mapping among the first sequencing data, the first DRS object, the TRS workflow and the WES run for all the samples submitted in the sequencer.

3. The workflow automation execution method applied in the cloud-native biomedical informatics platform of claim 2, wherein the sample-specific metadata comprises:
a first additional data (Read1_TRS_FQN) indicating how to associate the first DRS object to a first element of the at least one TRS_FQN;
wherein the first WES_FQN is generated based on the run identifier and the first additional data (Read1_TRS_FQN) and attached to the first DRS object, thereby establishing mapping among the first sequencing data, the first DRS object and the at least one TRS_FQN.

4. The workflow automation execution method applied in the cloud-native biomedical informatics platform of claim 3, wherein the sequencer further generates a second sequencing data, and the workflow automation execution method applied in the cloud-native biomedical informatics platform further comprises:
importing a second DRS object indicating the second sequencing data in response to the run sheet, wherein a second WES_FQN is generated based on the run identifier and the at least one TRS_FQN and attached to the second DRS object;
wherein the WES run is performed to run the TRS workflow on the first DRS object and the second DRS object in response to the run sheet, wherein the second DRS object is resolved based on the second WES_FQN;
wherein the sample-specific metadata further comprises a second additional data (Read2_TRS_FQN) indicating how to associate the second DRS object to a second element of the at least one TRS_FQN.

5. The workflow automation execution method applied in the cloud-native biomedical informatics platform of claim 4, wherein the sample-specific metadata further comprises:
a run name data (Run_Name) associating the first DRS object and the second DRS object to the WES run by assigning a unique run name based on the run sheet;
a workflow uniform resource locator data (Workflow_URL) specifying a TRS object to be used for the WES run; and
a runtime data (Runtimes) specifying a WES execution runtimes configuration in a format of key-value pairs of workflow description language (WDL) call-name and WES runtime options.

6. The workflow automation execution method applied in the cloud-native biomedical informatics platform of claim 5, further comprising:
obtaining a piece of the run sheet indicating a piece of the first sequencing data;
in response to a sample identification field of the run sheet, identifying a uniform resource identifier (URI) describing the piece of the first sequencing data;
in response to the unique run name and the at least one TRS_FQN of the run sheet, creating the first WES_FQN; and
creating the first DRS object associating the URI and the first WES_FQN.

7. The workflow automation execution method applied in the cloud-native biomedical informatics platform of claim 6, further comprising:
making a copy of the first sequencing data on a storage; and
creating the URI indicating the copy of the first sequencing data.

8. The workflow automation execution method applied in the cloud-native biomedical informatics platform of claim 5, further comprising:
in response to a run event, creating a WES object, wherein the run event indicates the run name data (Run_Name) and the TRS object;
checking whether the TRS object includes an auto-matching TRS_FQN;
in response to the run name data (Run_Name) and the auto-matching TRS_FQN, creating the first WES_FQN;
finding the first DRS object based on the first WES_FQN, wherein the first DRS object comprises a uniform resource identifier (URI); and
matching the URI to the auto-matching TRS_FQN.

9. The workflow automation execution method applied in the cloud-native biomedical informatics platform of claim 8, further comprising:
checking whether the TRS object includes a runtime resolving TRS_FQN indicating a value of the runtime resolving TRS_FQN is resolved based on the sample-specific metadata of a source TRS_FQN;
finding the first DRS object corresponding to the source TRS_FQN; and
resolving the value of the runtime resolving TRS_FQN based on the sample-specific metadata of the source TRS_FQN.

10. A workflow automation execution system, wherein a tool register service (TRS) workflow is registered in a cloud-native biomedical informatics platform, and the TRS workflow comprises at least one tool register service fully qualified name (TRS_FQN), and the workflow automation execution system comprising:
a memory storing a run sheet and a first sequencing data; and
a processor signally connected to the memory and configured to perform a workflow automation execution method applied in the cloud-native biomedical informatics platform, wherein the workflow automation execution method applied in the cloud-native biomedical informatics platform comprises:
providing the run sheet used to configure a sequencer to generate the first sequencing data, wherein the run sheet comprises a sample-specific metadata associating the first sequencing data, the at least one TRS_FQN and a run identifier;
importing a first data repository service (DRS) object indicating the first sequencing data in response to the run sheet, wherein a first workflow execution service fully qualified name (WES_FQN) is generated based on the run identifier and the at least one TRS_FQN and attached to the first DRS object; and
performing a workflow execution service (WES) run running the TRS workflow on the first DRS object in response to the run sheet, wherein the first DRS object is resolved based on the first WES_FQN.

11. The workflow automation execution system of claim 10, wherein the run sheet is a comma separated values (CSV) file and further comprises:
a header section comprising informational fields describing a context around which a sequencing run or analysis is performed; and
a sample section comprising the sample-specific metadata corresponding to each of a plurality of samples submitted in the sequencer;
wherein the run sheet is configured to specify mapping among the first sequencing data, the first DRS object, the TRS workflow and the WES run for all the samples submitted in the sequencer.

12. The workflow automation execution system of claim 11, wherein the sample-specific metadata comprises:
a first additional data (Read1_TRS_FQN) indicating how to associate the first DRS object to a first element of the at least one TRS_FQN;
wherein the first WES_FQN is generated based on the run identifier and the first additional data (Read1_TRS_FQN) and attached to the first DRS object, thereby establishing mapping among the first sequencing data, the first DRS object and the at least one TRS_FQN.

13. The workflow automation execution system of claim 12, wherein the sequencer further generates a second sequencing data, and the workflow automation execution method applied in the cloud-native biomedical informatics platform further comprises:
importing a second DRS object indicating the second sequencing data in response to the run sheet, wherein a second WES_FQN is generated based on the run identifier and the at least one TRS_FQN and attached to the second DRS object;
wherein the WES run is performed to run the TRS workflow on the first DRS object and the second DRS object in response to the run sheet, wherein the second DRS object is resolved based on the second WES_FQN;
wherein the sample-specific metadata further comprises a second additional data (Read2_TRS_FQN) indicating how to associate the second DRS object to a second element of the at least one TRS_FQN.

14. The workflow automation execution system of claim 13, wherein the sample-specific metadata further comprises:
a run name data (Run_Name) associating the first DRS object and the second DRS object to the WES run by assigning a unique run name based on the run sheet;
a workflow uniform resource locator data (Workflow_URL) specifying a TRS object to be used for the WES run; and
a runtime data (Runtimes) specifying a WES execution runtimes configuration in a format of key-value pairs of workflow description language (WDL) call-name and WES runtime options.

15. The workflow automation execution system of claim 14, wherein the workflow automation execution method applied in the cloud-native biomedical informatics platform further comprises:
obtaining a piece of the run sheet indicating a piece of the first sequencing data;
in response to a sample identification field of the run sheet, identifying a uniform resource identifier (URI) describing the piece of the first sequencing data;
in response to the unique run name and the at least one TRS_FQN of the run sheet, creating the first WES_FQN; and
creating the first DRS object associating the URI and the first WES_FQN.

16. The workflow automation execution system of claim 15, further comprising:
a storage signally connected to the processor, wherein the workflow automation execution method applied in the cloud-native biomedical informatics platform further comprises:
making a copy of the first sequencing data on the storage; and
creating the URI indicating the copy of the first sequencing data.

17. The workflow automation execution system of claim 14, wherein the workflow automation execution method applied in the cloud-native biomedical informatics platform further comprises:
in response to a run event, creating a WES object, wherein the run event indicates the run name data (Run_Name) and the TRS object;
checking whether the TRS object includes an auto-matching TRS_FQN;
in response to the run name data (Run_Name) and the auto-matching TRS_FQN, creating the first WES_FQN;
finding the first DRS object based on the first WES_FQN, wherein the first DRS object comprises a uniform resource identifier (URI); and
matching the URI to the auto-matching TRS_FQN.

18. The workflow automation execution system of claim 17, wherein the workflow automation execution method applied in the cloud-native biomedical informatics platform further comprises:
checking whether the TRS object includes a runtime resolving TRS_FQN indicating a value of the runtime resolving TRS_FQN is resolved based on the sample-specific metadata of a source TRS_FQN;
finding the first DRS object corresponding to the source TRS_FQN; and
resolving the value of the runtime resolving TRS_FQN based on the sample-specific metadata of the source TRS_FQN.

19. A computer readable medium having instructions therein, when executed, causing a processor to perform a workflow automation execution method applied in a cloud-native biomedical informatics platform, wherein a tool register service (TRS) workflow is registered in the cloud-native biomedical informatics platform, and the TRS workflow comprises at least one tool register service fully qualified name (TRS_FQN), and the workflow automation execution method applied in the cloud-native biomedical informatics platform comprising:
providing a run sheet used to configure a sequencer to generate a first sequencing data, wherein the run sheet comprises a sample-specific metadata associating the first sequencing data, the at least one TRS_FQN and a run identifier;
importing a first data repository service (DRS) object indicating the first sequencing data in response to the run sheet, wherein a first workflow execution service fully qualified name (WES_FQN) is generated based on the run identifier and the at least one TRS_FQN and attached to the first DRS object; and
performing a workflow execution service (WES) run running the TRS workflow on the first DRS object in response to the run sheet, wherein the first DRS object is resolved based on the first WES_FQN.

20. The computer readable medium of claim 19, wherein the run sheet is a comma separated values (CSV) file and further comprises:
a header section comprising informational fields describing a context around which a sequencing run or analysis is performed; and
a sample section comprising the sample-specific metadata corresponding to each of a plurality of samples submitted in the sequencer;
wherein the sample-specific metadata comprises:
a first additional data (Read1_TRS_FQN) indicating how to associate the first DRS object to a first element of the at least one TRS_FQN;
wherein the run sheet is configured to specify mapping among the first sequencing data, the first DRS object, the TRS workflow and the WES run for all the samples submitted in the sequencer, and the first WES_FQN is generated based on the run identifier and the first additional data (Read1_TRS_FQN) and attached to the first DRS object, thereby establishing mapping among the first sequencing data, the first DRS object and the at least one TRS_FQN.

21. The computer readable medium of claim 20, wherein the sequencer further generates a second sequencing data, and the workflow automation execution method applied in the cloud-native biomedical informatics platform further comprises:
importing a second DRS object indicating the second sequencing data in response to the run sheet, wherein a second WES_FQN is generated based on the run identifier and the at least one TRS_FQN and attached to the second DRS object;
wherein the WES run is performed to run the TRS workflow on the first DRS object and the second DRS object in response to the run sheet, wherein the second DRS object is resolved based on the second WES_FQN;
wherein the sample-specific metadata further comprises a second additional data (Read2_TRS_FQN) indicating how to associate the second DRS object to a second element of the at least one TRS_FQN.
